(19) United States
(12) Patent Application Publication
(10) Pub. No.: US 2022/0022956 A1
(43) Pub. Date: Jan. 27, 2022

(54) ENDOSCOPE SNARE

(71) Applicant: OLYMPUS CORPORATION, Tokyo (JP)

(72) Inventors: Noriaki YAMANAKA, Tokyo (JP); Noriko KOHIRA, Tokyo (JP); Yuji SAKAI, Kodaira-shi (JP); Seiji IWASAKI, Tokyo (JP)

(73) Assignee: OLYMPUS CORPORATION, Tokyo (JP)

(21) Appl. No.: 17/494,290

(22) Filed: Oct. 5, 2021

Related U.S. Application Data
(63) Continuation of application No. PCT/JP2019/016113, filed on Apr. 15, 2019.

(51) Int. Cl. A61B 18/14

(52) U.S. Cl. CPC A61B 18/1492; A61B 2018/00982

(57) ABSTRACT

An endoscope snare includes a sheath, a support wire inserted into the sheath and movable advance and retraction in the sheath along an axis, a loop-shaped conductive wire connected to a distal end of the support wire, and a pressing wire inserted into the sheath, connected to the conductive wire at a position different from that of the support wire, and movable advance and retraction in the sheath independently of the support wire. The pressing wire may have a plurality of wires, and may be connected to at least the conductive wire at two points of the support wire between which the axis is interposed.

This application is a continuation application based on a PCT International Application No. PCT/JP2019/016113, filed on Apr. 15, 2019. The content of the PCT International Application is incorporated herein by reference.

### BACKGROUND OF THE INVENTION

### Field of the Invention

The present invention relates to an endoscope snare.

### Description of Related Art

In the related art, in a treatment of early malignant tumors, for example, procedures such as endoscopic mucosal resection (EMR) and endoscopic submucosal dissection (ESD) are performed, which transendoscopically excise a lesion generated on a mucous membrane in a luminal organ such as a digestive tract. An endoscope snare, which is a high-frequency incision tool, is used as an endoscope treatment tool for excising a lesion tissue.

The endoscope snare has a loop-shaped snare loop at a distal end thereof.

When excising a lesion tissue with an endoscope snare, an operator needs to apply a high-frequency current to the snare loop in a state of sufficiently pressing a loop portion of the endoscope snare against the lesion tissue.

In an endoscope snare described in Japanese Unexamined Patent Application, First Publication No. 2006-95146, a middle portion of a snare loop is formed in a curved convex shape, and the snare loop is pressed against a lesion tissue to be excised so as not to slip.

### SUMMARY OF THE INVENTION

In order to solve the above problems, the present invention proposes the following means.

According to an aspect of the present invention, there is provided an endoscope snare including: a sheath; a support wire inserted into the sheath and movable advance and retraction along an axis in the sheath; a loop-shaped conductive wire connected to a distal end of the support wire; and a pressing wire inserted into the sheath, connected to the conductive wire at a position different from that of the support wire, and movable advance and retraction in the sheath independently of the support wire.

### BRIEF DESCRIPTION OF THE DRAWINGS

FIG. 1 is an overall view of an endoscope device used together with an endoscope snare according to a first embodiment.

FIG. 2 is a cross-sectional view of the endoscope snare in a long axis direction.

FIG. 3 is a perspective view of a conductive wire and a pressing wire protruding from a sheath of the endoscope snare.

FIG. 4 is a plan view of the conductive wire and the pressing wire of the endoscope snare.

FIG. 5 is a side view of the conductive wire and the pressing wire of the endoscope snare.

FIG. 6 is a side view of the conductive wire curved in a pressing direction.

FIG. 7 is a side view of the conductive wire caught in a lesion portion.

FIG. 8 is a side view of the conductive wire curved in the pressing direction.

FIG. 9 is a plan view showing a modification example of the pressing wire.

FIG. 10 is a plan view showing a modification example of the pressing wire.

FIG. 11 is a plan view showing a modification example of the conductive wire.

FIG. 12 is a plan view showing a modification example of the conductive wire.

FIG. 13 is a plan view showing a modification example of the pressing wire.

FIG. 14 is a cross-sectional view of a sheath containing a modification example of the conductive wire and the pressing wire.

FIG. 15 is a plan view showing a modification example of the pressing wire.

FIG. 16 is a perspective view of a conductive wire and a pressing wire of an endoscope snare according to a second embodiment.

FIG. 17 is a plan view of the conductive wire and the pressing wire of the endoscope snare.

### DETAILED DESCRIPTION OF THE INVENTION

### First Embodiment

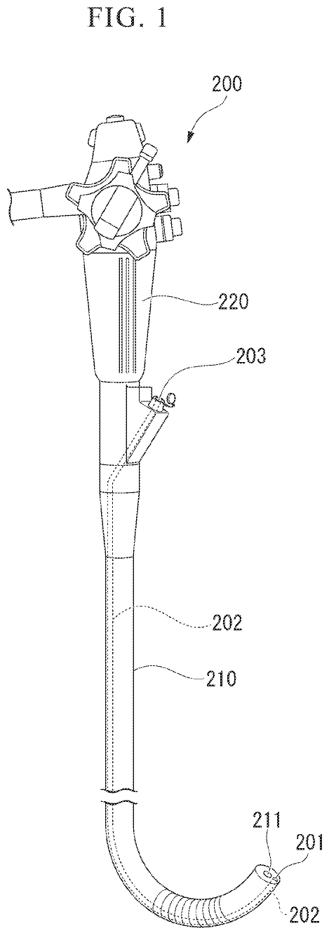

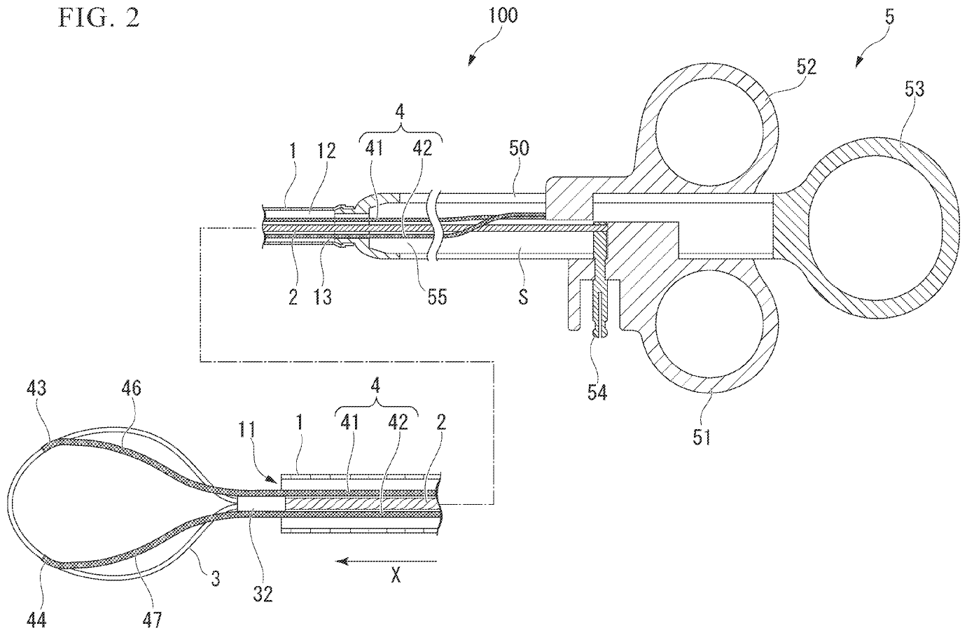

The endoscope snare 100 according to a first embodiment of the present invention will be described with reference to FIGS. 1 to 8. FIG. 1 is an overall view of an endoscope device 200 used together with the endoscope snare 100 according to the present embodiment. FIG. 2 is a cross-sectional view of the endoscope snare 100 in a long axis direction.

### [Endoscope Treatment System 300]

The endoscope treatment system 300 includes the endoscope snare 100 and the endoscope device 200. The endoscope snare 100 is inserted into a treatment tool channel 202 formed in an endoscope insertion portion 210 of the endoscope device 200.

As shown in FIG. 1, the endoscope device 200 is a known endoscope device having a treatment tool channel 202.

The endoscope device 200 includes the endoscope insertion portion 210 inserted into a body cavity, an endoscope operation unit 220 provided at a proximal end of the endoscope insertion portion 210, and an imaging unit 211 provided at a distal end of the endoscope insertion portion 210. A distal end opening 201 of the treatment tool channel 202 is opened at a distal end portion of the endoscope insertion portion 210. The treatment tool channel 202 is a passage extending from the distal end opening 201 to the entire length of the endoscope insertion portion 210, and a proximal end portion thereof is connected to a forceps opening 203 provided in the endoscope operation unit 220.

[Endoscope snare 100]

As shown in FIG. 2, the endoscope snare 100 includes a sheath 1, a support wire 2, a conductive wire 3, a pressing wire 4, and an operation unit 5. In the following description, a side of the operation unit 5 of the endoscope snare 100 is referred to as a proximal end side, and a side of the sheath 1 opposite to the operation unit 5 in a long axis direction X is referred to as a distal end side.

The sheath 1 is a long member that extends along the long axis direction X and can be inserted into the body cavity. The sheath 1 is made of an insulating material, for example, a fluororesin such as polytetrafluoroethylene (PTFB). The sheath 1 is flexible and is configured to be removable from the treatment tool channel 202 of the endoscope device 200 that meanders along a curved shape such as a lumen tissue in the body cavity.

A lumen 12 is formed in the sheath 1 over the entire length, and the sheath 1 has a distal end opening 11 and a proximal end opening 13 through which the lumen 12 communicates.

The support wire 2 is a stranded metal wire, and is inserted into the lumen 12 of the sheath 1 so as to be movable advance and retraction along an axis A of the support wire 2. A proximal end portion of the support wire 2 is connected to the operation unit 5.

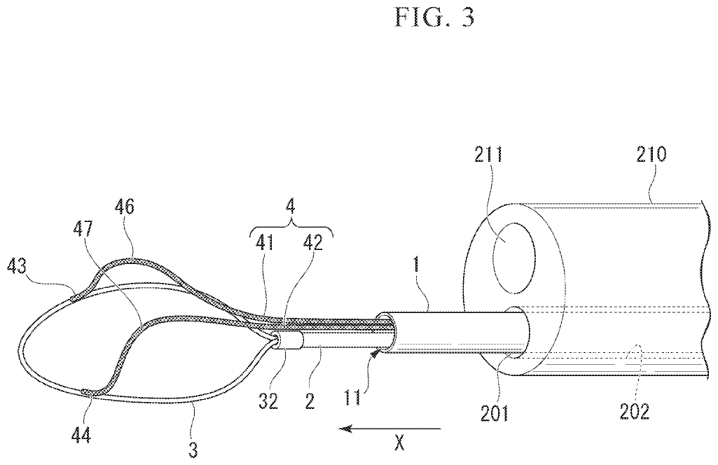

FIG. 3 is a perspective view of the conductive wire 3 and the pressing wire 4 protruding from the sheath 1.

The conductive wire 3 is a stranded metal wire connected to a distal end portion of the support wire 2, and is a snare loop formed in a loop shape. The conductive wire 3 functions as a monopolar electrode, and can incise an abutted lesion tissue by applying a high-frequency current to the conductive wire 3. The conductive wire 3 is formed of a stranded wire, and is easily caught in the lesion tissue to be incised and arranged.

The conductive wire 3 has flexibility, and when the conductive wire 3 is accommodated inside through the distal end opening 11 of the lumen 12 of the sheath 1, the conductive wire 3 is deformed into a shape that can be accommodated inside the lumen 12. When the conductive wire 3 protrudes toward the distal end side from the distal end opening 11 of the lumen 12 of the sheath 1, the conductive wire 3 returns to the original loop shape as shown in FIG. 3.

As shown in FIG. 2, the support wire 2 and the conductive wire 3 are connected by the coupling member 32 being brought into close contact with the outside, or by laser welding or brazing. The coupling member 32 is a tubular member made of metal.

The pressing wire 4 has two single metal wires (first pressing wire 41 and second pressing wire 42), and is inserted into the lumen 12 of the sheath 1 so as to be movable advance and retraction. The pressing wire 4 can move advance and retraction in the lumen 12 independently of the support wire 2. A proximal end portion of the pressing wire 4 is connected to the operation unit 5.

A distal end portion of the first pressing wire 41 and the conductive wire 3 are connected at a first connection portion 43 by a caulking member or laser welding. An outer peripheral portion of the first pressing wire 41 except for the first connection portion 43 is covered with an insulator such as rubber.

A distal end portion of the second pressing wire 42 and the conductive wire 3 are connected at a second connection portion 44 by a caulking member or laser welding. An outer peripheral portion of the second pressing wire 42 except for the second connection portion 44 is covered with an insulator such as rubber.

The conductive wire 3 and the pressing wire 4 may be connected by brazing, but it is desirable that the conductive wire 3 and the pressing wire 4 are connected by a caulking member or laser welding that does not generate unclean substances in order to enable assembly in a crane room.

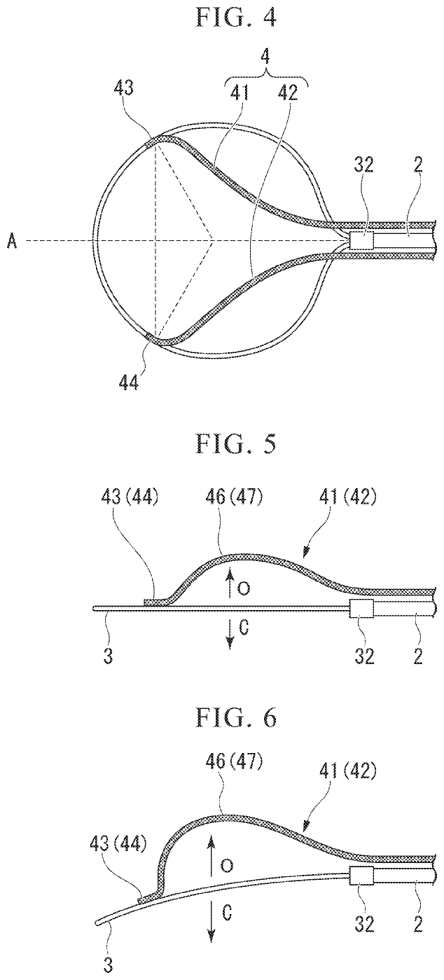

FIG. 4 is a plan view of the conductive wire 3 and the pressing wire 4.

The first connection portion 43 and the second connection portion 44 are disposed at positions facing each other on both sides in a state where the axis A of the support wire 2 is interposed therebetween. Further, the first connection portion 43, the second connection portion 44, and the coupling member 32 are disposed at positions where a loop shape of the conductive wire 3 is substantially equally divided in a plan view.

The proximal end portions of the first pressing wire 41 and the second pressing wire 42 are connected to the operation unit 5. The first pressing wire 41 and the second pressing wire 42 may be twisted from an intermediate portion located between the distal end portion and the proximal end portion to the proximal end portion.

FIG. 5 is a side view of the conductive wire 3 and the pressing wire 4.

The first pressing wire 41 has a first bent portion 46 that is raised in an out-of-plane direction O with respect to a surface P formed by the loop shape of the conductive wire 3 in the vicinity of the first connection portion 43.

The second pressing wire 42 has a second bent portion 47 that is raised in the out-of-plane direction O with respect to the surface P formed by the loop shape of the conductive wire 3 in the vicinity of the second connection portion 44.

FIG. 6 is a side view of the conductive wire 3 curved in a pressing direction C.

When the pressing wire 4 is moved forward with respect to the conductive wire 3, the conductive wire 3 is curved in a direction (hereinafter referred to as “pressing direction C”) opposite to the out-of-plane direction O in which the first bent portion 46 and the second bent portion 47 are raised. Since a bending rigidity of the pressing wire 4 is larger than a bending rigidity of the conductive wire 3, when the pressing wire 4 is moved forward with respect to the conductive wire 3, the conductive wire 3 is curved more than the pressing wire 4.

As shown in FIG. 2, the operation unit 5 includes an operation unit body 50 connected to the proximal end portion of the sheath 1, a first slider 51, a second slider 52, a handle 53, and a power supply connector 54.

As shown in FIG. 2, the operation unit body 50 has an internal space S into which the support wire 2 and the pressing wire 4 can be inserted. A distal end opening 55 of the internal space S communicates with the proximal end opening 13 of the sheath 1. The support wire 2 and the pressing wire 4 pass through the proximal end opening 13 of the sheath 1 and the distal end opening 55 of the internal space S and extend to the internal space S.

The first slider 51 is attached to the operation unit body 50 to be movable in the long axis direction X of the sheath 1. A proximal end portion of the support wire 2 is connected to the first slider 51. When the operator moves the first slider 51 advance and retraction relatively with respect to the operation unit body 50, the support wire 2 moves advance and retraction with respect to the sheath 1.

When the operator moves the first slider 51 forward relatively with respect to the operation unit body 50, the conductive wire 3 protrudes from the distal end opening 11 of the sheath 1. Further, when the operator moves the first slider 51 rearward relatively with respect to the operation unit body 50, the conductive wire 3 is accommodated inside the sheath 1.

The second slider 52 is attached to the operation unit body 50 to be movable in the long axis direction X of the sheath 1. The first slider 51 and the second slider 52 can be operated independently of the operation unit body 50. A proximal end portion of the pressing wire 4 is connected to the second slider 52. When the operator moves the second slider 52 advance and retraction relatively with respect to the operation unit body 50, the pressing wire 4 moves advance and retraction with respect to the sheath 1.

The handle 53 is a member fixed to the operation unit body 50. The operator holds the first slider 51, the second slider 52, and the handle 53 to perform the procedure.

The power supply connector 54 can be connected to a high-frequency power supply device (not shown), and is electrically and physically connected to the proximal end portion of the support wire 2. The power supply connector 54 can supply a high-frequency current supplied from the high-frequency power supply device to the conductive wire 3 via the support wire 2.

### [Operation of Endoscope Treatment System 300]

Next, an operation of the endoscope treatment system 300 of the present embodiment will be described. The operation of the endoscope treatment system 300 will be described by taking as an example a procedure of making a full-circumference incision in a lesion portion (early cancer or the like) P formed in a large intestine using the endoscope treatment system 300. The procedure to which the endoscope treatment system 300 is applied is not limited to this. For example, the endoscope treatment system 300 is also applied to a procedure for excising a portion of a lesion portion P or the like.

As a preparatory work, the operator identifies the lesion portion P by a known method and bulges the lesion portion P. Specifically, the operator inserts the endoscope insertion portion 210 of the endoscope device 200 into the large intestine, and identifies the lesion portion P while observing the image obtained by the imaging unit 211 of the endoscope. Next, the operator inserts a known submucosal local injection needle (not shown) into the treatment tool channel 202 of the endoscope insertion portion 210, and injects a liquid for local injection (local injection liquid) between the lesion portion P and a muscular layer W3 by the submucosal local injection needle to bulge the lesion portion P. After the operator injects the local injection liquid, the operator removes the submucosal local injection needle from the treatment tool channel 202.

The operator inserts the endoscope snare 100 into the treatment tool channel 202, and protrudes the distal end portion of the sheath 1 from the distal end opening 201 of the endoscope insertion portion 210. When the endoscope snare 100 is inserted into the treatment tool channel 202, the conductive wire 3 is accommodated inside the sheath 1. The operator protrudes the distal end portion of the sheath 1 to the vicinity of the lesion portion P while checking an endoscopic image.

The operator moves the first slider 51 and the second slider 52 of the operation unit 5 forward relatively with respect to the operation unit body 50, and simultaneously protrudes the conductive wire 3 and the pressing wire 4 from the distal end opening 11 of the sheath 1. The operator arranges the conductive wire 3 opened in a loop shape so that the pressing direction C faces the lesion portion P. The operator appropriately move the endoscope insertion portion 210 or the sheath 1 advance and retraction so that the conductive wire 3 is caught in the lesion portion P.

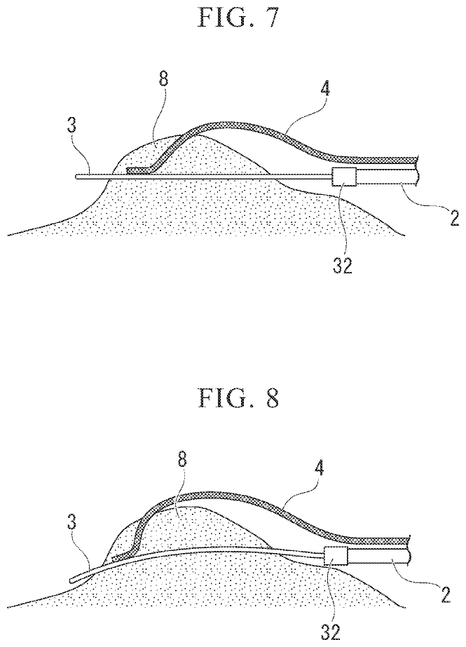

FIG. 7 is a side view of the conductive wire 3 caught in the lesion portion P.

As shown in FIG. 7, the conductive wire 3 is caught in the lesion portion P, but is not firmly in contact with a mucosal layer W around the lesion portion P over the entire periphery of the lesion portion P. When a high-frequency current flows through the conductive wire 3 in this state, there is a possibility that a portion of the mucosal layer W around the lesion portion P may not be incised.

FIG. 8 is a side view of the conductive wire 3 curved in the pressing direction C.

The operator does not move the first slider 51 relatively with respect to the operation unit body 50, but moves only the second slider 52 forward relatively with respect to the operation unit body 50 to move the pressing wire 4 forward with respect to the conductive wire 3. The conductive wire 3 is curved in the pressing direction C opposite to the out-of-plane direction O in which the first bent portion 46 and the second bent portion 47 are raised. As a result, the conductive wire 3 is pressed around the lesion portion P, and the entire periphery of the conductive wire 3 is firmly in contact with the mucosal layer W1 around the lesion portion P.

The operator supplies a high-frequency current to the power supply connector 54, and causes the high-frequency current to flow from the conductive wire 3 to the mucosal layer W1 around the lesion portion P. At the same time, the operator moves the first slider 51 rearward relatively with respect to the operation unit body 50 to perform an incision on the entire periphery of the lesion portion P.

Since the pressing wire 4 is covered with an insulator, when a high-frequency current flows through the conductive wire 3, even in a case where the pressing wire 4 is in contact with a tissue other than the lesion portion P to be incised, the high-frequency current does not flow through the tissue with which the pressing wire 4 is in contact.

According to the endoscope snare 100 according to the first embodiment, the operator can easily press the conductive wire 3 against the lesion portion P to be excised by moving the pressing wire 4 forward with respect to the conductive wire 3. The first pressing wire 41 and the second pressing wire 42 are connected to the conductive wire 3 at positions facing each other on both sides in a state where the axis A of the support wire 2 is interposed therebetween (first connection portion 43 and second connection portion 44). Therefore, by moving the pressing wire 4 forward with respect to the conductive wire 3, the pressing wire 4 can uniformly press the conductive wire 3 in the pressing direction C without twisting. Further, as compared with a case where the pressing wire is connected on the axis A of the support wire 2, it is easier to maintain the loop shape of the conductive wire 3 when the pressing wire 4 is moved advance and retraction with respect to the conductive wire 3.

According to the endoscope snare 100 according to the first embodiment, by moving the pressing wire 4 forward with respect to the conductive wire 3 in a state where the distal end portion of the conductive wire 3 is caught in the lesion portion P, the entire periphery of the conductive wire 3 is firmly in contact with the mucosal layer W1 around the lesion portion P. The pressing wire 4 has the first bent portion 46 and the second bent portion 47 that are raised in the out-of-plane direction O opposite to the pressing direction C, and the conductive wire 3 can be easily pressed in the pressing direction C.

According to the endoscope snare 100 according to the first embodiment, the first connection portion 43, the second connection portion 44, and the coupling member 32 are each disposed at positions where the loop shape of the conductive wire 3 is substantially equally divided in a plan view. Since the coupling member 32 functions as a fulcrum and the first connection portion 43 and the second connection portion 44 function as action points, the pressing wire 4 easily presses the conductive wire 3 in the pressing direction C.

As described above, the first embodiment of the present invention is described in detail with reference to the drawings. However, specific configurations are not limited to the embodiment, and include a design modification or the like within a scope which does not depart from the gist of the present invention. In addition, components shown in the above-described embodiment and modification examples shown below can be appropriately combined and configured.

### Modification Example 1

In the above embodiment, the pressing wire 4 has the first bent portion 46 and the second bent portion 47, but an aspect of the pressing wire is not limited to this. The pressing wire may not have the first bent portion 46 and the second bent portion 47. Even when the pressing wire does not have the first bent portion 46 and the second bent portion 47, the pressing wire can press the conductive wire 3 by moving the pressing wire forward with respect to the conductive wire 3.

### Modification Example 2

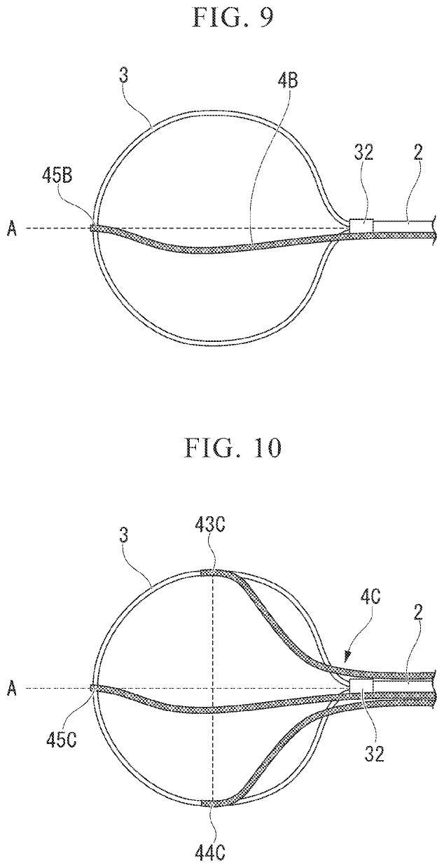

In the above embodiment, the pressing wire 4 is two wires, but the aspect of the pressing wire is not limited to this. FIG. 9 is a plan view showing a pressing wire 4B which is a modification example of the pressing wire. The pressing wire 4B is a single metal wire, and is connected to the conductive wire 3 on the axis A of the support wire 2. A portion of the pressing wire 4B except for a connection portion 45B with the conductive wire 3 is covered with an insulator. By moving the pressing wire 4B forward with respect to the conductive wire 3, the pressing wire 4B can press the conductive wire 3 in the pressing direction C.

### Modification Example 3

In the above embodiment, the pressing wire 4 is two wires, but the aspect of the pressing wire is not limited to this. FIG. 10 is a plan view showing a pressing wire 4C which is a modification example of the pressing wire. The pressing wire 4C is three metal wires. The pressing wire 4C is connected to the conductive wire 3 at a first connection portion 43C and a second connection portion 44C disposed at positions facing each other on both sides in a state where the axis A of the support wire 2 is interposed therebetween, and a third connection portion 45C disposed on the axis A of the support wire 2. The first connection portion 43C, the second connection portion 44C, the third connection portion 45C, and the coupling member 32 are disposed at positions where the loop shape of the conductive wire 3 is substantially equally divided in a plan view. In the pressing wire 4C, at least two connection portions (first connection portion 43C and second connection portion 44C) are disposed at positions facing each other on both sides in the state where the axis A of the support wire 2 is interposed therebetween, and by moving the pressing wire 4C forward with respect to the conductive wire 3, the pressing wire 4C can easily press the conductive wire 3 in the pressing direction C without twisting. The pressing wire 4C has a larger number of wires than the pressing wire 4, and can more reliably transmit a force for pressing the conductive wire 3 in the pressing direction C.

### Modification Example 4

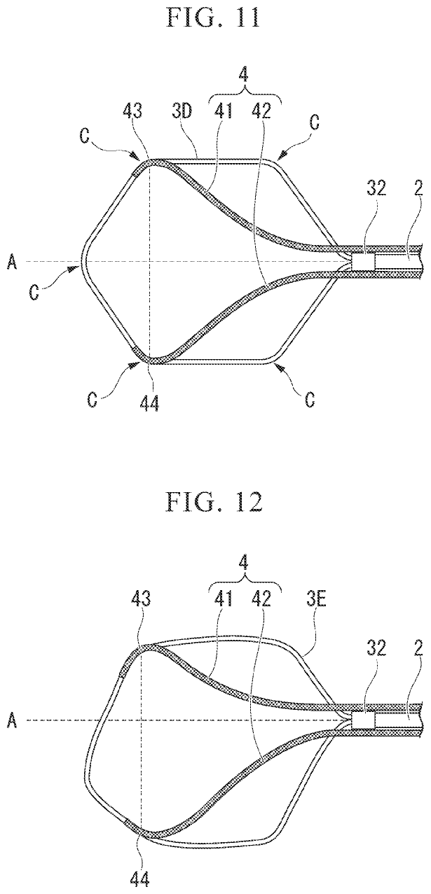

In the above embodiment, the conductive wire 3 is formed in a circular loop shape having no corner portions, but the aspect of the conductive wire is not limited to this. FIG. 11 is a plan view showing a conductive wire 3D which is a modification example of the conductive wire. The conductive wire 3D has five corner portions C, and is formed in a substantially hexagonal shape in a plan view. The first connection portion 43 and the second connection portion 44 are provided at the corner portions C of the conductive wire 3D. Therefore, the pressing wire 4 can uniformly press the conductive wire 3D in the pressing direction C without twisting. Further, when the pressing wire 4 is attached to the conductive wire 3D, an attachment position can be easily grasped, and manufacturing variations of the attachment position and the like can be reduced.

### Modification Example 5

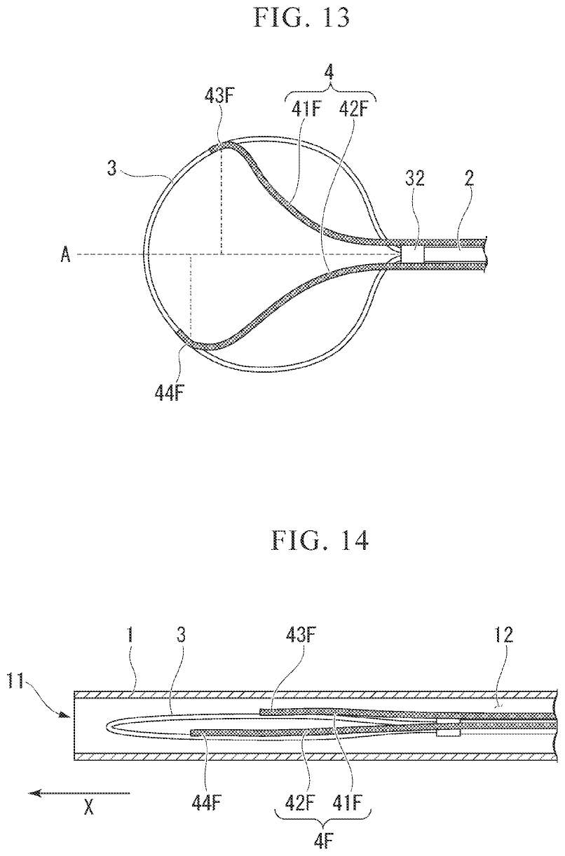

In the above embodiment, the conductive wire 3 is formed in a shape symmetrical with respect to the axis A of the support wire 2, but the aspect of the conductive wire is not limited to this. FIG. 12 is a plan view showing a conductive wire 3E which is a modification example of the conductive wire. The conductive wire 3E is formed in a shape asymmetrical with respect to the axis A of the support wire 2. Even when the conductive wire 3E is formed in an asymmetrical shape with respect to the axis A, by moving the pressing wire 4 forward with respect to the conductive wire 3E, the pressing wire 4 can press the conductive wire 3E in the pressing direction C. The first connection portion 43 and the second connection portion 44 are disposed at positions facing each other on both sides in the state where the axis A of the support wire 2 is interposed therebetween, and the pressing wire 4 can easily press the conductive wire 3E in the pressing direction C without twisting.

### Modification Example 6

In the above embodiment, the pressing wire 4 is connected to the conductive wire 3 at positions facing each other on both sides in the state where the axis A of the support wire 2 is interposed therebetween, but the aspect of the pressing wire is not limited to this. FIG. 13 is a plan view showing a pressing wire 4F which is a modification example of the pressing wire. The pressing wire 4F is two single metal wires (first pressing wire 41F and second pressing wire 42F), and is connected to the conductive wire 3 at positions which do not face each other on both sides in a state where the axis A of the support wire 2 is interposed. The first connection portion 43F, which is the connection portion between the conductive wire 3 and the first pressing wire 41F, and the second connection portion 44F, which is the connection portion between the conductive wire 3 and the second pressing wire 42F, are disposed at positions (different positions) shifted in the direction in which the axis A of the support wire 2 extends.

FIG. 14 is a cross-sectional view of the sheath 1 accommodating the conductive wire 3 and the pressing wire 4F.

When the conductive wire 3 and the pressing wire 4F are accommodated inside the lumen 12 from the distal end opening 11, the first connection portion 43F and the second connection portion 44F do not overlap in the long axis direction X of the sheath 1. Accordingly, the conductive wire 3 and the pressing wire 4F can be easily accommodated inside the lumen 12.

### Modification Example 7

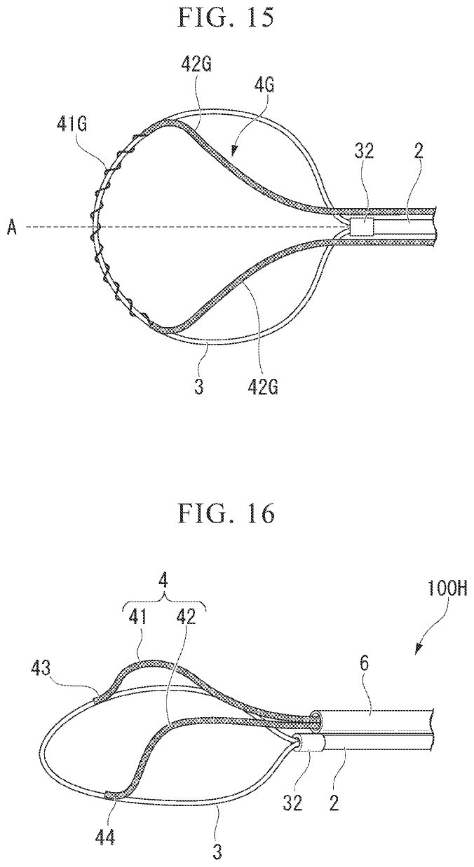

In the above embodiment, the pressing wire 4 is two wires, but the aspect of the pressing wire is not limited to this. FIG. 15 is a plan view showing a pressing wire 4G which is a modification example of the pressing wire. The pressing wire 4G is a single metal wire, and has a conductive portion 41G in which a conductor is exposed and a covering portion 42G in which the conductor is covered. The conductive portion 41G is formed at an intermediate position interposed between the covering portions 42G. In the pressing wire 4G, the conductive portion 41G is wound around a distal end portion of the conductive wire 3 and fixed. Both end portions of the pressing wire 4G are attached to the second slider 52. By moving the pressing wire 4G forward with respect to the conductive wire 3, the pressing wire 4G can press the conductive wire 3 in the pressing direction C. The pressing wire 4G increases a rigidity of the distal end portion of the conductive wire 3, and unevenness is formed on the distal end portion. Accordingly, the distal end portion of the conductive wire 3 is less likely to slip on the tissue.

### Modification Example 8

In the above embodiment, the conductive wire 3 is formed of a stranded metal wire and the pressing wire 4 is formed of a single metal wire, but the aspects of the conductive wire and the pressing wire are not limited thereto. The conductive wire may be formed of a single metal wire. The pressing wire may be formed of a stranded metal wire. Further, the pressing wire may be made of resin, and in this case, the pressing wire is fixed by the conductive wire and the caulking member.

### Second Embodiment

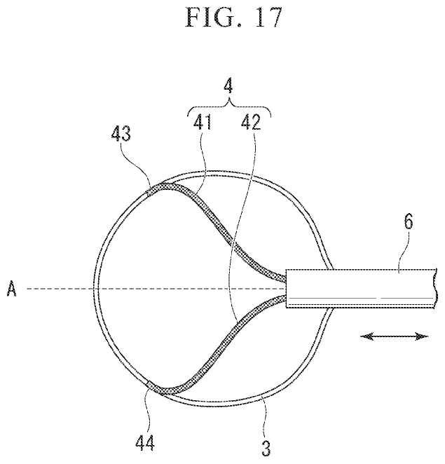

An endoscope snare 100H according to a second embodiment of the present invention will be described with reference to FIGS. 16 and 17. In the following description, the same components as those already described are denoted by the same reference numerals, and repeated descriptions will be omitted. FIG. 16 is a perspective view of the conductive wire 3 and the pressing wire 4 of the endoscope snare 100H. FIG. 17 is a plan view of the conductive wire 3 and the pressing wire 4 of the endoscope snare 100H.

### [Endoscope Snare 100H]

The endoscope snare 100H includes the sheath 1, the support wire 2, the conductive wire 3, the pressing wire 4, the operation unit 5, and a advance/retraction movement tube 6.

As shown in FIG. 16, the advance/retraction movement tube 6 is a tubular member into which the pressing wire 4 is inserted, and is inserted into the lumen 12 of the sheath 1 so as to be movable advance and retraction. The advance/retraction movement tube 6 can be moved advance and retraction in the long axis direction X of the sheath 1 by the operation unit 5.

Next, the operation of the endoscope snare 100H of the present embodiment will be described.

The operator moves the second slider 52 forward relatively with respect to the operation unit body 50, and moves the advance/retraction movement tube 6 forward in the long axis direction X of the sheath 1 before moving the pressing wire 4 forward with respect to the conductive wire 3. After that, the operator moves only the second slider 52 forward relatively with respect to the operation unit body 50, and moves the pressing wire 4 forward with respect to the conductive wire 3. The conductive wire 3 is curved in the pressing direction C opposite to the out-of-plane direction O in which the first bent portion 46 and the second bent portion 47 are raised. As a result, the conductive wire 3 is pressed around the lesion portion P, and the entire periphery of the conductive wire 3 is firmly in contact with the mucosal layer W1 around the lesion portion P.

In a case where the advance/retraction movement tube 6 is moved forward in the long axis direction X of the sheath 1, as shown in FIG. 17, when the pressing wire 4 is pressed, a fulcrum of a pressing force moves forward, a force component in a direction of opening the loop shape of the conductive wire 3 becomes large, and the loop shape of the conductive wire 3 becomes difficult to close.

According to the endoscope snare 100H according to the present embodiment, the conductive wire 3 can be easily pressed against the lesion portion P to be excised while maintaining the loop shape open.

As described above, the second embodiment of the present invention is described in detail with reference to the drawings. However, specific configurations are not limited to the embodiment, and include a design modification or the like within a scope which does not depart from the gist of the present invention. In addition, components shown in the above-described embodiment and modification examples shown below can be appropriately combined and configured.

What is claimed is:

1. An endoscope snare comprising:
a sheath;
a support wire inserted into the sheath and movable advance and retraction in the sheath along an axis;
a loop-shaped conductive wire connected to a distal end of the support wire; and
a pressing wire inserted into the sheath, connected to the conductive wire at a position different from that of the support wire, movable advance and retraction in the sheath independently of the support wire, and capable of biasing the conductive wire in a direction intersecting a plane direction of the conductive wire.

2. The endoscope snare according to claim 1,
wherein the pressing wire has a plurality of wires, and
at least two of the plurality of wires of the pressing wire are connected to the conductive wire at two points of the support wire between which the axis is interposed.

3. The endoscope snare according to claim 1,
wherein the pressing wire has a bent portion that is raised in an out-of-plane direction with respect to a surface formed by the loop shape of the conductive wire.

4. The endoscope snare according to claim 1,
wherein bending rigidity of the pressing wire is larger than bending rigidity of the conductive wire.

5. The endoscope snare according to claim 4,
wherein the conductive wire is a stranded wire, and
the pressing wire is a single wire.

6. The endoscope snare according to claim 1,
wherein the pressing wire has a plurality of wires, and
the plurality of wires are movable advance and retraction independently.

7. The endoscope snare according to claim 1,
wherein a connection portion to which the pressing wire and the conductive wire are connected and a coupling member to which the support wire and the conductive wire are coupled are disposed at positions where the loop shape of the conductive wire is substantially equally divided in a plan view.

8. The endoscope snare according to claim 1,
wherein at least a portion of the conductive wire has a corner portion, and
the pressing wire is connected to the corner portion.

9. The endoscope snare according to claim 1,
wherein the conductive wire and the pressing wire are connected to each other by a caulking member or laser welding.

10. The endoscope snare according to claim 1,
wherein the pressing wire has a plurality of wires,
the conductive wire and the pressing wire are connected to each other by a caulking member, and
a plurality of the caulking members are disposed at different positions in an axial direction of the support wire.

11. The endoscope snare according to claim 1,
wherein two pressing wires are provided.
